(19) United States
(12) Patent Application Publication
(10) Pub. No.: US 2021/0300153 A1
(43) Pub. Date: Sep. 30, 2021

(54) AIR CURTAIN SYSTEM

(71) Applicant: DENSO International America, Inc., Southfield, MI (US)

(72) Inventors: Matthew Michael PORTER, Farmington Hills, MI (US); Claudia AMBRIZ, Commerce Twp., MI (US); Shogo KONDO, Kariya-city (JP); Carolina RIGNEY, West Bloomfield, MI (US); Young Kwon LEE, Canton, MI (US)

(21) Appl. No.: 16/836,042

(22) Filed: Mar. 31, 2020

(51) Int. Cl. B60H 1/24; B60H 3/00; B60H 3/02; B60H 3/06; B60H 1/22; B60H 1/32; A61L 9/14

(52) U.S. Cl. CPC B60H 1/245; B60H 3/0028; B60H 3/0078; B60H 3/02; A61L 2209/134; B60H 1/2225; B60H 1/3227; A61L 9/14; A61L 2209/16; B60H 3/0608

(57) ABSTRACT

An air conditioning system for a vehicle including an air curtain assembly. The air curtain assembly includes an outer blower configured to generate an air curtain, and an inner blower configured to generate comfort airflow within the air curtain. The air conditioning system is configured to condition the comfort airflow with respect to at least one of the following: temperature, humidity, odor, particulate matter content, biological matter content, germ content, and infectious agent content.

### FIELD

The present disclosure relates to an air curtain system, such as for a vehicle.

### BACKGROUND

This section provides background information related to the present disclosure, which is not necessarily prior art.

While current vehicle cabin comfort systems are suitable for their intended use, they are subject to improvement. For example, when using a ride sharing service, two or more passengers unfamiliar with one another may be seated next to each other for an extended period of time. The passengers may have different preferences regarding climate control. Furthermore, the passengers may be sensitive to odors emanating from the other passengers, such as from perfume or cologne, for example. Isolation of each passenger from germs or other contagions would also be desirable. The present disclosure advantageously includes an air conditioning system that addresses these issues, and provides numerous additional advantages and unexpected results as explained in detailed herein, and as one skilled in the art will appreciate.

### SUMMARY

This section provides a general summary of the disclosure, and is not a comprehensive disclosure of its full scope or all of its features.

The present disclosure provides for an air conditioning system for a vehicle including an air curtain assembly. The air curtain assembly includes an outer blower configured to generate an air curtain, and an inner blower configured to generate comfort airflow within the air curtain. The air conditioning system is configured to condition the comfort airflow with respect to at least one of the following: temperature, humidity, odor, particulate matter content, biological matter content, germ content, and infectious agent content.

The present disclosure further provides for an air conditioning system for a vehicle including a first air curtain assembly at a first seat of the vehicle and a second air curtain assembly at a second seat of the vehicle. A control module is configured to control first comfort airflow to the first air curtain assembly and second comfort airflow to the second air curtain assembly such that the first comfort airflow and the second comfort airflow differ with respect to at least one of the following: temperature, humidity, odor, particulate matter content, biological matter content, germ content, and infectious agent content.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of select embodiments and not all possible implementations, and are not intended to limit the scope of the present disclosure.

FIG. 1 is a side view of an example of an air conditioning system in accordance with the present disclosure installed in a vehicle;

FIG. 2 is a top view of FIG. 1;

FIG. 3 illustrates an example of an air curtain of the air conditioning system of FIGS. 1 and 2;

FIG. 4 illustrates various components of the air conditioning system of the present disclosure, including an HVAC unit, humidifier, ionizer, air filter, fragrance atomizer, and airflow distribution case;

FIG. 5 illustrates a control module of the air conditioning system of the present disclosure, and a user interface for operating the air conditioning system;

FIG. 6A illustrates an example of control of the HVAC system by the control module;

FIG. 6B is a continuation of FIG. 6A; and

FIG. 7 illustrates an example of a system for activating and configuring the air conditioning system.

Corresponding reference numerals indicate corresponding parts throughout the several views of the drawings.

### DETAILED DESCRIPTION

Example embodiments will now be described more fully with reference to the accompanying drawings.

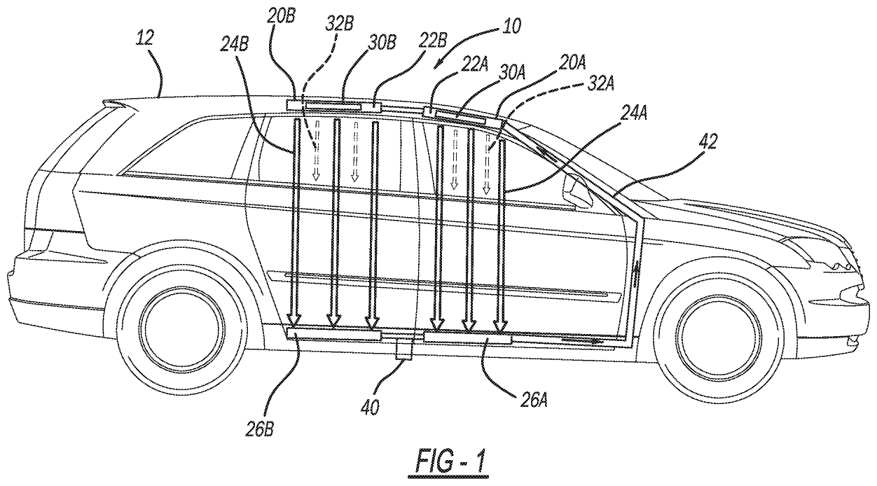

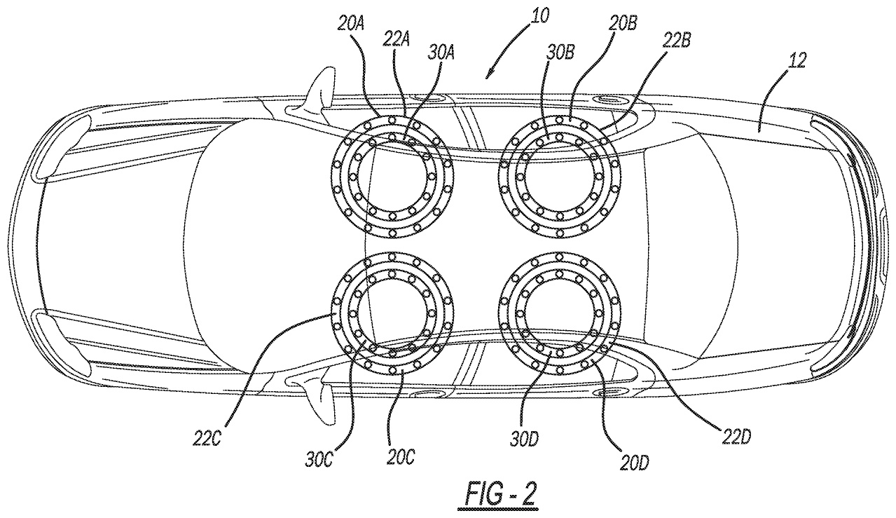

FIGS. 1 and 2 illustrate one example of an air conditioning system 10 in accordance with the present disclosure. Although the air conditioning system 10 is illustrated as installed in an example of a passenger vehicle 12, the air conditioning system 10 may be installed in any other suitable vehicle as well. Other possible vehicles include any other type of passenger vehicle, mass transit vehicle, construction vehicle/equipment, military vehicle, school bus, train, tram, aircraft, watercraft, etc. The air conditioning system 10 may also be used with any suitable non-vehicular application as well. For example, the air conditioning system 10 may be installed in a movie theatre, conference hall, lecture hall, classroom, stadium, concert hall, etc.

The air conditioning system 10 includes a plurality of air curtain assemblies for generating individual air curtains about two or more occupants of the vehicle 12. For example, the air conditioning system 10 may include a first air curtain assembly 20A, a second air curtain assembly 20B, a third air curtain assembly 20C, and a fourth air curtain assembly 20D. Each one of the air curtain assemblies 20A-20D is at a different seat of the vehicle 12. Although four air curtain assemblies 20A-20D are illustrated, the vehicle 12 may include any suitable number of air curtain assemblies at any suitable locations about the vehicle 12.

The first air curtain assembly 20A includes an outer blower 22A, which generates an outer air curtain 24A. The outer blower 22A is circular and includes a plurality of air vents through which airflow passes to generate the outer air curtain 24A, which is generally circular and received by receiving vent 26A, which is also circular. The outer air curtain 24A provides a barrier isolating an occupant seated under the first air curtain assembly 20A from other occupants of the vehicle 12 and airflow outside of the outer air curtain 24A.

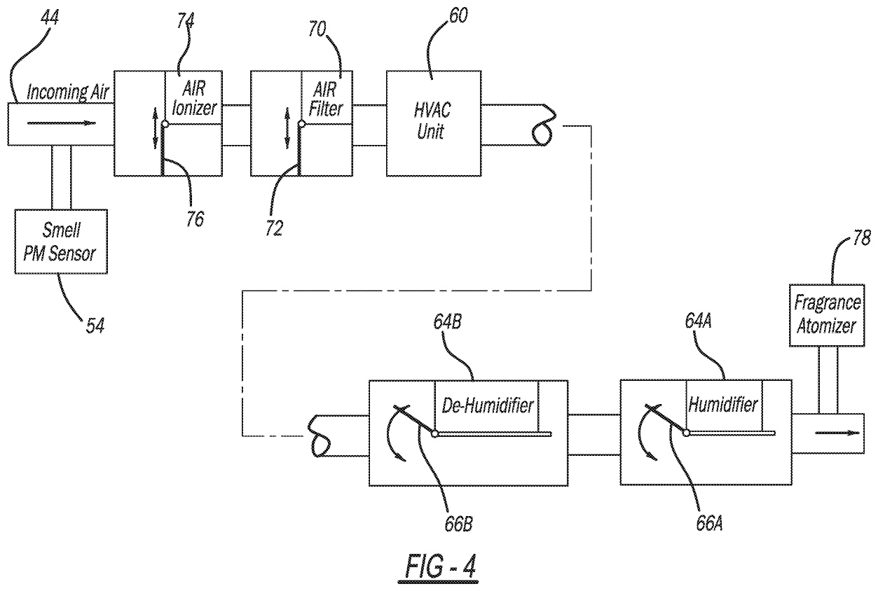

The outer blower 22A surrounds an inner blower 30A. The inner blower 30A is generally circular and includes a plurality of outlets through which airflow passes to generate comfort air 32A, which flows inside of the outer air curtain 24A. The comfort air 32A is conditioned by the air conditioning system 10 as described herein to enhance passenger comfort. Airflow to the inner blower 30A flows through air duct 44, as illustrated in FIG. 4. Airflow to the inner blower 30A is conditioned by the elements of FIG. 4 to provide the comfort air 32A, as described further herein.

An external vent 40 is included to vent the air curtain assemblies 20A-20D. An air duct 42 is in fluid communication with the receiving vents 26A, 26B (along with receiving vents of the air curtain assemblies 20C, 20D) and the outer blowers 22A-22D to circulate airflow between the receiving vents 26A, 26B (along with receiving vents of the air curtain assemblies 20C, 20D) and the outer blowers 22A-22D. The air duct 42 may include an air inlet for allowing external air to enter the air conditioning system 10.

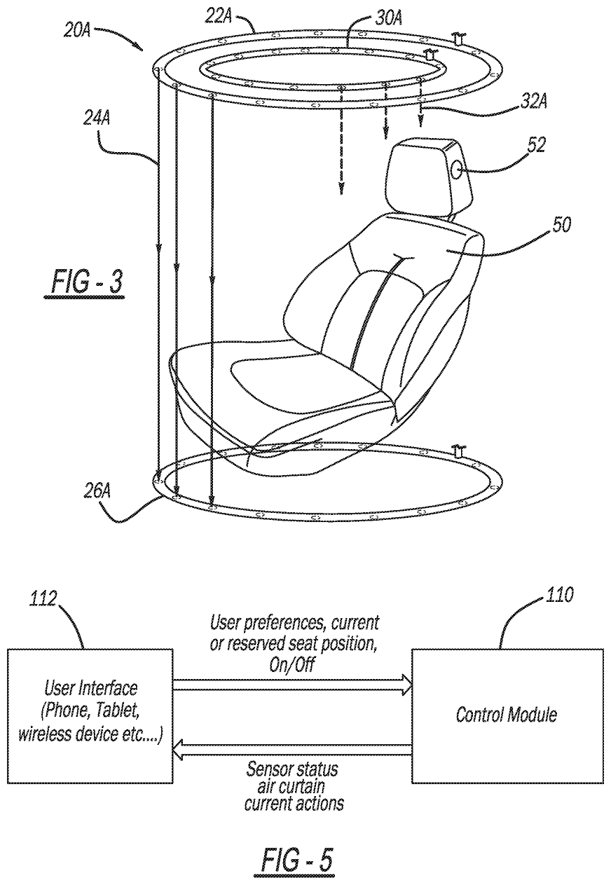

As illustrated in FIG. 3, the first air curtain assembly 20A surrounds a seat 50 of the vehicle 12. Thus, the first air curtain assembly 20A isolates the occupant of the seat 50 from airflow outside of the air curtain 24A and delivers comfort air 32A to the occupant of the seat 50. The comfort air 32A is conditioned by the components of FIG. 4.

The air conditioning system 10 includes one or more sensors for sensing air conditions throughout the air conditioning system 10. For example and as illustrated in FIG. 3, the seat 50 includes one or more sensors 52. The sensors 52 may be any sensors suitable for measuring temperature, humidity, presence of bacteria, pollen, dander, particulate matter, and/or odors, for example. The sensors 52 may be located at a head rest of the seat 50 or at any other suitable location. An smell/particulate matter sensor 54 may also be arranged along the air duct 44 of FIG. 4.

The air curtain assemblies 20B, 20C, and 20D are each substantially similar to, or the same as, the first air conditioning system 20A. The features of the air curtain assemblies 20B, 20C, and 20D are designated throughout the drawings using the same reference numerals as the first air conditioning system 20A, but with the suffixes “B,” “C,” and “D” corresponding to the air curtain assemblies 20B, 20C, and 20D. With respect to the common features, the description of the first air curtain assembly 20A also applies to the air curtain assemblies 20B, 20C, and 20D.

With particular reference to FIG. 4, airflow to the inner blower 30A through the air duct 44 passes through an HVAC unit 60. The HVAC unit 60 is configured to heat and/or cool airflow passing therethrough. Thus, the HVAC unit 60 includes any suitable evaporator and/or suitable heater. Suitable heaters include, but are not limited to, a heater core or PTC heater, for example. Airflow from the HVAC unit 60 may be directed through a humidifier 64A or a dehumidifier 64B to adjust the humidity of the airflow. Airflow control doors 66A and 66B are movable to direct airflow through or around the humidifier 64A and the dehumidifier 64B.

Along the air duct 44 upstream of the HVAC unit 60 is an air filter 70 and an air ionizer 74, which may be configured to filter air in any suitable manner, such as for dirt, dust, particulate matter, germs, odors, biological matter, infectious agents including viruses, etc. Airflow through the air filter 70 is controlled by any suitable airflow control door 72, for example. Airflow through the air ionizer 74 is controlled by any suitable airflow control door 76. Along the air duct 44 may also be any suitable fragrance atomizer 78. The fragrance atomizer 78 may be downstream of the humidifier 64A, for example.

FIG. 4 illustrates an air duct 44, which extends to the inner blower 30A. However, the present disclosure further includes a plurality of additional air ducts each extending to a different one of the inner blowers 30B, 30C, and 30D. Each one of the additional air ducts has an HVAC unit, air filter, air ionizer, humidifier, de-humidifier, fragrance atomizer, and smell/PM sensor similar to that illustrated in FIG. 4 and described above. As a result, comfort air to each of the inner blowers 30A-30D may be customized for each seat and occupant of the vehicle 12.

Alternatively, the system 10 may include a plurality of additional air ducts extending through the elements of FIG. 4 to each one of the different inner blowers 30B, 30C, and 30D of the different air curtain assemblies 20B, 20C, and 20D to provide comfort air within the air curtains of the air curtain assemblies 20B-20D. Thus, although only the doors 66A, 66B, 72, and 76 of the air duct 44 are illustrated, additional doors may be included for each one of the additional air ducts extending to the different inner blowers 30B, 30C, 30D. Furthermore, the HVAC unit 60 may be configured to accommodate the separate air ducts and individually condition the airflow in each air duct to different temperatures by including a plurality of airflow control doors.

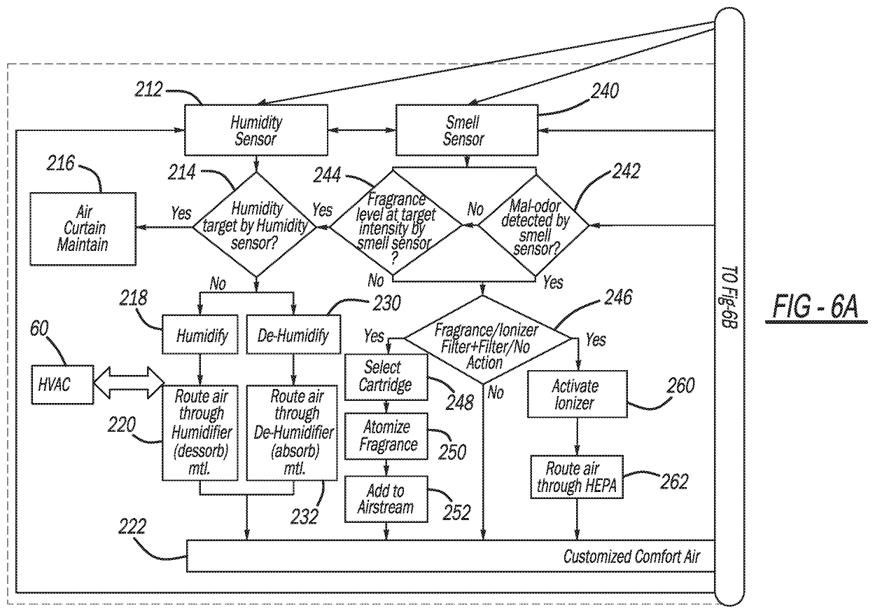

FIG. 5 illustrates the control module 110 in communication with any suitable user interface 112, such as a smartphone, tablet, wireless device, or dashboard controls of the vehicle 12. In this application, including the definitions below, the term “control module” may be replaced with the term “circuit.” The term “control module” may refer to, be part of, or include processor hardware (shared, dedicated, or group) that executes code and memory hardware (shared, dedicated, or group) that stores code executed by the processor hardware. The code is configured to provide the features of the control module 110 described herein.

The control module 110 is in receipt of any suitable commands from the user interface 112, such as activation/deactivation commands, user preferences, current or reserved seat position, temperature inputs, etc. The control module 110 transmits to the user interface 112 the operational state of the air curtain assemblies 20A-20D, sensor readings, and any other suitable information.

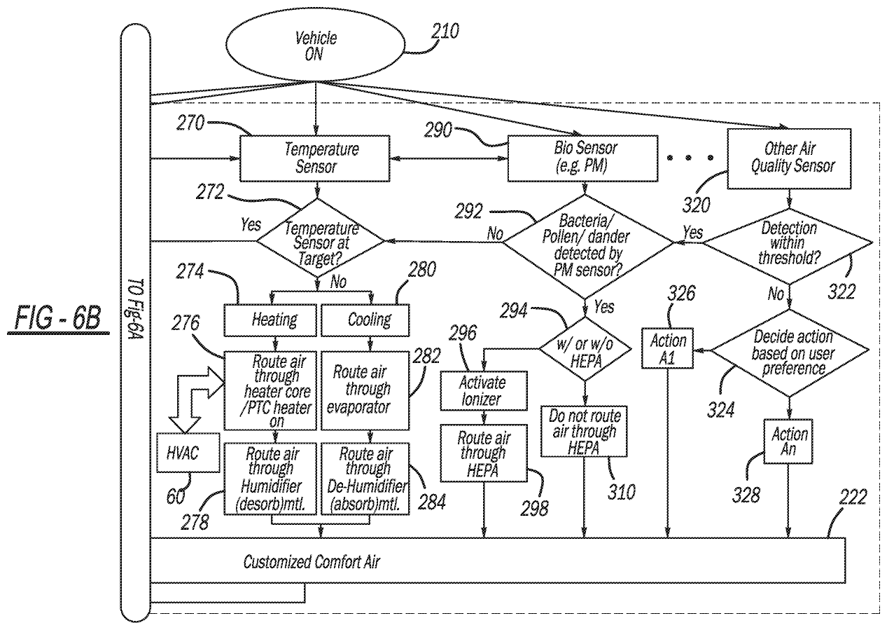

FIGS. 6A and 6B illustrate an example of operation of the air conditioning system 10 by the control module 110 in accordance with the present disclosure. FIGS. 6A and 6B will be described herein with respect to the first air curtain assembly 20A at the seat 50. However, the control module 110 is likewise configured to operate each one of the air curtain assemblies 20B-20D in a similar manner, and thus the following description of the operation of the first air curtain assembly 20A also applies to operation of each one of the air curtain assemblies 20B-20D. Thus, the temperature, air filtration, air ionization, fragrances, etc. may be customized at each one of the air curtain assemblies 20A-20D.

Starting at block 210, when the vehicle 12 is turned on, the control module 110 receives inputs from various sensors about the vehicle 12, such as the temperature and humidity sensors 52, and the smell and particulate matter sensor 54. Specifically and with reference to block 212 of FIG. 6A, the control module 110 receives an input from the humidity sensor 52 indicating the humidity at the first seat 50.

At block 214, the control module 110 determines whether the humidity is at a set humidity target. If the humidity is at the set target, the control module 110 proceeds to block 216 where the current humidity settings of the comfort air 32A are maintained. If at block 214, the measured humidity is not at the set humidity target, the control module 110 proceeds to block 218 if the humidity is below the target or block 230 if the humidity is above the target. At block 218, the control module 110 increases the humidity of the comfort air 32A in any suitable manner, such by directing the air through the humidifier 64A (humidity desorption material) at block 220. The control module 110 may also operate the HVAC unit 60 to increase the humidity. If the humidity of the comfort air 32A is above the humidity target, at block 230 the control module 110 dehumidifies the air, such as by routing the air through the dehumidifier 64B (humidity absorption material) at block 232. From blocks 220 and 232, the control module 110 proceeds to block 222 where the comfort air 32A flows to the seat 50 at the set humidity level.

At block 240, the control module 110 receives inputs from the odor/PM sensor 54. At block 242, the control module 110 determines whether a malicious odor is present based on the input from the smell/PM sensor 54. If no malicious odor is detected, the control module 110 proceeds to block 244. At block 244, the control module 110 determines whether a fragrance level of the airflow is at a target intensity based on the input from the smell/PM sensor 54. If the fragrance level is at the target intensity, then the control module 110 proceeds to block 214. If at block 242 the control module 110 determines that a malicious odor is present, then the control module 110 proceeds to block 246. Likewise, if at block 244 the control module 110 determines based on the input of the smell sensor 54 that the fragrance level is not at the target intensity, the control module 110 proceeds to block 246.

At block 246, the control module 110 determines whether comfort air 32A should be filtered and/or ionized based on blocks 242 and 244. If the control module 110 determines that comfort air 32A does not need to be filtered or ionized, the control module 110 proceeds to block 222. If use of the fragrance atomizer 78 is called for, the control module 110 proceeds to block 248 where the control module 110 selects an appropriate fragrance cartridge. At block 250, the fragrance cartridge is atomized, and at block 252, the fragrance is added to the comfort air 32A. From block 252, the control module 110 proceeds to block 222.

If at block 246 the control module 110 determines that comfort air 32A should be ionized and/or filtered, the control module 110 proceeds to block 260 where the control module 110 activates the air ionizer 74 and positions the airflow control door 76 to direct airflow through the air ionizer 74. From block 260, the control module 110 proceeds to block 262 where the control module 110 operates the airflow control door 72 to route airflow through the air filter 70. The filter 70 may be any suitable air filter, such as a high efficiency particulate air (HEPA) filter. From block 262, the control module 110 proceeds to block 222.

At block 270, the control module 110 receives inputs from the temperature sensor 52 identifying the temperature at the seat 50. At block 272, the control module 110 determines whether the current temperature is at a set target temperature. If the temperature is at the target temperature, the control module 110 proceeds to block 242. If the temperature is not at the set target temperature, the control module 110 proceeds to block 274 if heating is required, or proceeds to block 280 if cooling is required, to reach the target temperature. If heating is required, the control module 110 proceeds to block 276, and the control module 110 operates the HVAC unit 60 to heat the comfort air 32A, such as directing airflow across a heater core or activated PTC heater. At block 278, the control module 110 routes the comfort air 32A through the humidifier 64A. From block 278, the control module 110 proceeds to block 222.

If cooling is required to reach the set target temperature, the control module 110 proceeds from block 280 to block 282 where the control module 110 operates the HVAC unit 60 to cool comfort air 32A, such as by routing the airflow across an evaporator of the HVAC unit 60. From block 282, the control module 110 proceeds to block 284, where the control module 110 directs the comfort air 32A through the dehumidifier 64B. From block 284, the control module 110 proceeds to block 222.

At block 290, the control module 110 receives inputs from the smell sensor/particulate matter sensor 54. From block 290, the control module 110 proceeds to block 292, where the control module 110 determines whether excessive bacteria, pollen and/or dander is detected by the smell/PM sensor 54. If excessive bacteria, pollen and/or dander is not detected, the control module 110 proceeds to block 272. If excessive bacteria, pollen and/or dander is detected, the control module 110 proceeds to block 294. At block 294, the control module 110 determines whether or not filtration by the air filter 70 is called for. If filtration by the air filter 70 is not called for, the control module 110 actuates airflow control door 72 to direct comfort air 32A around the air filter 70 at block 310. From block 310, the control module 110 proceeds to block 222. If at block 294 the control module 110 determines that airflow ionization and filtration is called for, the control module 110 proceeds to block 296 where the control module 110 activates the air ionizer 74 and actuates the airflow control door 76 to route airflow through the air ionizer. From block 296, the control module 110 proceeds to block 298, where the control module 110 actuates the airflow control door 72 to direct comfort air 32 through the air filter 70. From block 298, the control module 110 proceeds to block 222.

The air conditioning system 10 may also include any other suitable air quality sensors arranged at any suitable position about the vehicle 12. At block 320, the control module 110 receives inputs from such air quality sensors, and at block 322 the control module 110 determines whether the readings from those sensors are within predetermined thresholds. If the predetermined thresholds are not exceeded, the control module 110 proceeds to block 292. If the thresholds are exceeded, the control module 110 proceeds to block 324. At block 324, the control module 110 identifies appropriate action to be taken. Any suitable action may be taken, such as any suitable first action represented by “Action A1” at block 326. Any other suitable action may be taken, such as represented by “Action An” at block 328. From blocks 326 and 328, the control module 110 proceeds to block 222.

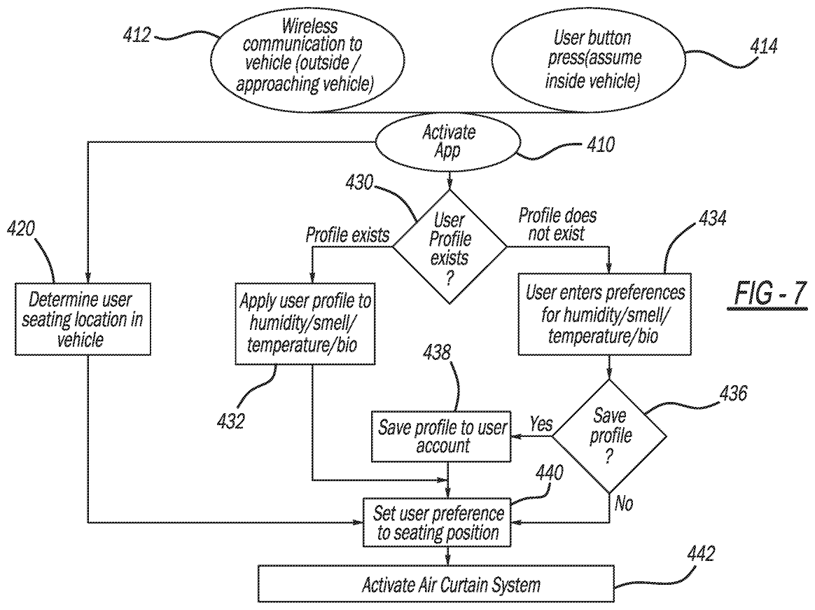

FIG. 7 illustrates operation of a personalization application for the air conditioning system 10. The personalization application may be included with the control module 110 or any other suitable device, such as a smartphone, tablet computer, personal computer, server accessible via the internet, etc. The application may be activated at block 410 in response to wireless communication with the control module 110 initiated by a user on his or her smartphone as the user is approaching the vehicle 12, at block 412. The application may also be activated in response to a user pressing a button on the dashboard of the vehicle 12 at block 414. After the application has been activated, the application will determine the seating location of the user within the vehicle at block 420. From block 420, the application proceeds to block 440 where the user's preferences are set based on the seating position. The application also determines whether or not a user profile exists at block 430. If a profile does exist, the application proceeds to block 432, where the application and control module 110 operate the HVAC unit 60, the humidifier/dehumidifier 64A, 64B, the air ionizer 74, the air filter 70 and the fragrance atomizer 78 in accordance with the user profile settings. From block 432, the application proceeds to block 440.

If a user profile does not exist, the application proceeds to block 434 where the user enters his or her preferences regarding humidity, smell, temperature, and biofiltration (for filtering particulate matter, germs, biological matter, infectious agents etc.), for example. At block 436, the user is given the option to save his or her profile. If the user wants to save his or her profile, the profile settings are saved at block 438, and then the application proceeds to block 440. If the user does not want his or her profile saved, the application proceeds directly to block 440 from block 436. From block 440 the application proceeds to block 442, where the control module 110 activates the air conditioning system 10 in accordance with the user's preferences.

The present disclosure thus advantageously provides for an air conditioning system 10 that allows each occupant of the vehicle 12 to customize various airflow settings at his or her seating position. For example, air curtains 24A and 24B isolate airflow between two seating positions. Comfort air 32A and 32B inside each one of the air curtains 24A, 24B, respectively, delivers airflow customized to the preferences of the occupants. Thus, in ride sharing vehicles in which passengers do not know one another, each passenger may isolate the airflow at his or her seat from the airflow of other occupants to increase the comfort of each occupant and reduce the likelihood that a particular occupant will be subject to undesirable odors, germs, viruses, etc. of other occupants of the vehicle 12. One skilled in the art will appreciate that the present disclosure provides numerous additional advantages and unexpected results.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

The terminology used herein is for the purpose of describing particular example embodiments only and is not intended to be limiting. As used herein, the singular forms “a,” “an,” and “the” may be intended to include the plural forms as well, unless the context clearly indicates otherwise. The terms “comprises,” “comprising,” “including,” and “having,” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

When an element or layer is referred to as being “on,” “engaged to,” “connected to,” or “coupled to” another element or layer, it may be directly on, engaged, connected or coupled to the other element or layer, or intervening elements or layers may be present. In contrast, when an element is referred to as being “directly on,” “directly engaged to,” “directly connected to,” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

What is claimed is:

1. An air conditioning system for a vehicle comprising:
an air curtain assembly including an outer blower configured to generate an air curtain and an inner blower configured to generate comfort airflow within the air curtain;
wherein the air conditioning system is configured to condition the comfort airflow with respect to at least one of the following: temperature, humidity, odor, particulate matter content, biological matter content, germ content, and infectious agent content.

2. The air conditioning system of claim 1, further comprising an HVAC unit configured to heat and cool the comfort airflow.

3. The air conditioning system of claim 1, further comprising an air filter configured to filter particulates from the comfort airflow.

4. The air conditioning system of claim 1, further comprising a humidifier configured to increase humidity of the comfort airflow.

5. The air conditioning system of claim 1, further comprising a de-humidifier configured to decrease humidity of the comfort airflow.

6. The air conditioning system of claim 1, further comprising a fragrance atomizer configured to add fragrance to the comfort airflow.

7. The air conditioning system of claim 1, wherein the outer blower surrounds the inner blower.

8. The air conditioning system of claim 1, further comprising a receiving vent configured to receive the air curtain.

9. The air conditioning system of claim 1, further comprising a control module configured to save user settings for the comfort airflow with respect to at least one of the following: temperature, humidity, fragrance, and filtration.

10. An air conditioning system for a vehicle comprising:
a first air curtain assembly at a first seat of the vehicle;
a second air curtain assembly at a second seat of the vehicle; and
a control module configured to control first comfort airflow to the first air curtain assembly and second comfort airflow to the second air curtain assembly such that the first comfort airflow and the second comfort airflow differ with respect to at least one of the following: temperature, humidity, odor, particulate matter content, biological matter content, germ content, and infectious agent content.

11. The air conditioning system of claim 10, further comprising at least one air ionizer configured to ionize at least one of the first comfort airflow and the second comfort airflow.

12. The air conditioning system of claim 10, further comprising at least one air filter configured to filter at least one of the first comfort airflow and the second comfort airflow.

13. The air conditioning system of claim 10, further comprising at least one humidifier configured to increase humidity of at least one of the first comfort airflow and the second comfort airflow.

14. The air conditioning system of claim 10, further comprising at least one de-humidifier configured to decrease humidity of at least one of the first comfort airflow and the second comfort airflow.

15. The air conditioning system of claim 10, further comprising at least one fragrance atomizer configured to add fragrance to at least one of the first comfort airflow and the second comfort airflow.

16. The air conditioning system of claim 10, further comprising at least one heating, ventilation, and air conditioning (HVAC) unit configured to heat and cool at least one of the first comfort airflow and the second comfort airflow.

17. The air conditioning system of claim 16, wherein the HVAC unit includes an evaporator and heater.

18. The air conditioning system of claim 16, wherein the HVAC unit further includes a blower.

19. The air conditioning system of claim 10, wherein the control module is configured to receive inputs from a remote electronic device, the inputs representative of user commands for operating the air conditioning system.

20. The air conditioning system of claim 10, wherein:
the first air curtain assembly is configured to generate a first air curtain surrounding the first comfort airflow; and
the second air curtain assembly is configured to generate a second air curtain surrounding the second comfort airflow.
